SEQUENCE LISTING (16 sequences)

SEQ ID NO: 1 - DNA, 20 bp, Artificial Sequence
  Description of Artificial Sequence Synthetic primer
gcacagtcaaggccgagaat

SEQ ID NO: 2 - DNA, 20 bp, Artificial Sequence
  Description of Artificial Sequence Synthetic primer
gccttctccatggtggtgaa

SEQ ID NO: 3 - DNA, 21 bp, Artificial Sequence
  Description of Artificial Sequence Synthetic primer
atggattcccgttcgagtacg

SEQ ID NO: 4 - DNA, 20 bp, Artificial Sequence
  Description of Artificial Sequence Synthetic primer
tcagctggatagcgacatcg

SEQ ID NO: 5 - DNA, 23 bp, Artificial Sequence
  Description of Artificial Sequence Synthetic primer
cacccttcttcatcccactctta

SEQ ID NO: 6 - DNA, 18 bp, Artificial Sequence
  Description of Artificial Sequence Synthetic primer
accaaggtggctgcatcc

SEQ ID NO: 7 - DNA, 17 bp, Artificial Sequence
  Description of Artificial Sequence Synthetic primer
tggctggaccagatgcg

SEQ ID NO: 8 - DNA, 25 bp, Artificial Sequence
  Description of Artificial Sequence Synthetic primer
gacgtgaatcttctgcttaacaaca

SEQ ID NO: 9 - DNA, 20 bp, Artificial Sequence
  Description of Artificial Sequence Synthetic primer
tgtgtgcttcgaaggctatg

SEQ ID NO: 10 - DNA, 20 bp, Artificial Sequence
  Description of Artificial Sequence Synthetic primer
gcagacacactcgttctcca

SEQ ID NO: 11 - DNA, 20 bp, Artificial Sequence
  Description of Artificial Sequence Synthetic primer
ctgacagaggcaccactgaa

SEQ ID NO: 12 - DNA, 20 bp, Artificial Sequence
  Description of Artificial Sequence Synthetic primer
catctccagagtccagcaca

SEQ ID NO: 13 - DNA, 21 bp, Artificial Sequence
  Description of Artificial Sequence Synthetic primer
aaagtgcatccggacacctaa

SEQ ID NO: 14 - DNA, 21 bp, Artificial Sequence
  Description of Artificial Sequence Synthetic primer
tgcagccagaaagctcaaact

SEQ ID NO: 15 - protein, 469 aa, Homo sapiens
MNDFGIKNMDQVAPVANSYRGTLKRQPAFDTFDGSLFAVFPSLNEEQTLQEVPTGLDSISHDSANCELPLLTPCSKAVMSQALKATFSGFKKEQRRLGIPKNPWLWSEQQVCQWLLWATNEFSLVNVNLQRFGMNGQMLCNLGKERFLELAPDFVGDILWEHLEQMIKENQEKTEDQYEENSHLTSVPHWINSNTLGFGTEQAPYGMQTQNYPKGGLLDSMCPASTPSVLSSEQEFQMFPKSRLSSVSVTYCSVSQDFPGSNLNLLTNNSGTPKDHDSPENGADSFESSDSLLQSWNSQSSLLDVQRVPSFESFEDDCSQSLCLNKPTMSFKDYIQERSDPVEQGKPVIPAAVLAGFTGSGPIQLWQFLLELLSDKSCQSFISWTGDGWEFKLADPDEVARRWGKRKNKPKMNYEKLSRGLRYYYDKNIIHKTSGKRYVYRFVCDLQNLLGFTPEELHAILGVQPDTED

SEQ ID NO: 16 - protein, 467 aa, Mus musculus
NDFGIKNMDQVAPVANSFRGTLKRQPAFDTFDGSLFAVLPSLSEDQTLQEVPTGLDSVSHDSASCELPLLTPCSKAVMSQALKATFSGFQKEQRRLGIPKNPWLWSEQQVCQWLLWATNEFSLVNVNLHQFGMNGQMLCNLGKERFLELAPDFVGDILWEHLEQMIKENQEKTEDQYEENSHLNAVPHWINSNTLGFSMEQAPYGMQAPNYPKDNLLDSMCPPSATPAALGSELQMLPKSRLNTVNVNYCSISQDFPSSNVNLLNNNSGKPKDHDSPENGGDSFESSDSLLRSWNSQSSLLDVQRVPSFESFEEDCSQSLCLSKLTMSFKDYIQERSDPVEQGKPVIPAAVLAGFTGSGPIQLWQFLLELLSDKSCQSFISWTGDGWEFKLADPDEVARRWGKRKNKPKMNYEKLSRGLRYYYDKNIIHKTSGKRYVYRFVCDLQNLLGFTPEELHAILGVQPDTED

(19) United States
(12) Patent Application Publication
(10) Pub. No.: US 2011/0280865 A1
(43) Pub. Date: Nov. 17, 2011

(54) Ets-2 Biomarkers for Fibrotic Diseases and Uses Thereof

(75) Inventors: Clay B. Marsh, Columbus, OH (US); Christopher P. Baran, Hilliard, OH (US)

(73) Assignee: THE OHIO STATE UNIVERSITY, Columbus, OH (US)

(21) Appl. No.: 13/144,578

(22) PCT Filed: Jan. 15, 2010
(86) PCT No.: PCT/US2010/021166

§ 371 Date: Jul. 28, 2011

Related U.S. Application Data
(60) Provisional application No. 61/145,198, filed on Jan. 16, 2009.

(51) Int. Cl. A61K 39/395; C12Q 1/68; C40B 40/06; C40B 30/04; A61P 37/00; C07K 16/00; A61K 31/7105; A61K 31/711; A61K 38/02; G01N 33/566; C07K 14/00

(52) U.S. Cl. 424/130.1; 436/501; 435/7.92; 435/6.12; 435/6.11; 506/16; 506/9; 530/350; 530/389.1; 514/44 A; 514/44 R; 514/1.1

(57) ABSTRACT

Methods and compositions for detecting, treating, characterizing, and diagnosing interstitial lung and/or fibrotic diseases are described.

### CROSS-REFERENCE TO RELATED APPLICATIONS

This application claims the benefit of U.S. Provisional Application Ser. No. 61/145,198 filed Jan. 16, 2009, the entire disclosure of which is expressly incorporated herein by reference.

### STATEMENT REGARDING FEDERALLY SPONSORED RESEARCH

This invention was made with government support under Grant Nos. R1 HL095431-01 and R01 HL067176, awarded by the National Institutes of Health. The government has certain rights in the invention.

### SEQUENCE LISTING

The instant application contains a Sequence Listing which has been submitted via EFS-web and is hereby incorporated by reference in its entirety. The ASCII copy, created on Jan. 12, 2010, is named 604_{—}50723_SEQ_LIST_OSURF-09078.txt, and is 11,518 bytes in size.

### TECHNICAL FIELD AND INDUSTRIAL APPLICABILITY OF THE INVENTION

The present invention relates to compositions and methods for detecting, treating, characterizing, and diagnosing interstitial lung and/or fibrotic diseases. More particularly, the present invention provides a novel marker useful for the diagnosis, characterization, and treatment of interstitial lung and/or fibrotic diseases. In particular, the present invention is directed to methods for determining which subjects will most benefit from treatment therapies that affect such markers.

### BACKGROUND OF THE INVENTION

Interstitial lung diseases are a broad set of diseases that perturb lung function by affecting the space between the endothelial cells of the vascular bed and the epithelial cells of the alveoli. In normal conditions, this interstitial space consists of a negligible amount of supportive matrix, thereby allowing the efficient transport of oxygen and carbon dioxide. Any increase or thickening of this space with cells, fluid, collagen or other materials acts to hamper gas exchange and causes functional lung abnormalities. One set of lung diseases characterized by such interstitial thickening and lung fibrosis is known as idiopathic interstitial pneumonias (IIPs).

In particular, one form of IIP is idiopathic pulmonary fibrosis (IPF), a progressive and untreatable form of interstitial lung disease. Despite years of exhaustive research into the underlying pathophysiological mechanisms, patients with IPF have a median survival of 3-5 years following diagnosis. For example, from 1992-2003, the mortality rates for patients with IPF significantly increased, despite the ongoing investigation into the molecular mechanisms of the disease. The only consistent treatment option is lung transplantation, with more than 30% of patients dying on the waiting list.

Human observational data and animal models have been used to study the underlying mechanisms of pulmonary fibrosis. For example, the inventors herein recently demonstrated that macrophage colony-stimulating factor (M-CSF) and alveolar macrophages are important in pulmonary fibrosis through the production and secretion of C-C chemokine ligand 2 (CCL2) and CCL12. The inventors herein have also found that CCL2 and CCL12 direct the recruitment of monocytes and fibrocytes, respectively, to the lung via the C-C chemokine receptor 2 (CCR2).

Thus, despite the growing body of knowledge regarding IPF, there is still a need in the art to uncover the identity and function of the genes involved in IPF pathogenesis.

There is also a need for reagents and assays to accurately detect IPF, to define various stages of disease progression, to identify and characterize genetic alterations defining such disease onset and progression, and to treat and prevent IPF.

### SUMMARY

The present invention is based in part on the discovery of a role for the transcription factor ets-2 in the pathogenesis of pulmonary fibrosis.

In one aspect, there is provided a novel marker useful for detecting interstitial fibrotic disorders, and other fibrosing organ diseases, such as in the lung.

In another aspect, there is provided herein novel pharmaceutical targets and genetic markers for fibrotic diseases, methods for modifying such biomarkers, and uses of the same.

In another aspect, there is provided herein a method of treating different types of fibrosis-related disorders, such as lung diseases characterized by interstitial thickening and lung fibrosis. It would be especially useful to have methods of treating fibrosis-related disorders that modulate the transcription factors that mediate fibrosis.

In another aspect, there is provided herein a method of screening compounds that are capable of modulating activation of the transcription factor ets-2 response, especially methods that modulate the expression of the transcription factors involved in the response.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIGS. 1A-1D: Ets-2 (A72/A72) mice have a decreased response to bleomycin-induced pulmonary fibrosis. Ets-2 (WT/WT) and ets-2 (A72/A72) mice were treated with bleomycin (0.035 U/g) or PBS (vehicle control) as described in Materials and Methods.

FIG. 1A: Following sacrifice, the lungs from bleomycin treated mice were formalin fixed, sectioned, and stained with H&E (top panels) or trichrome (bottom panels). Data is representative of at least 3 mice per condition.

FIG. 1B: Mouse lungs were also used to quantitate collagen content using the Sircol collagen assay. Data represents n=4 for ets-2 (WT/WT)+PBS; n=5 for ets-2 (A72/A72)+PBS; n=6 ets-2 (WT/WT)+bleomycin; n=6 for ets-2 (A72/A72)+bleomycin.

FIGS. 1C and 1D: RNA from mouse lungs was also isolated, and mRNA was amplified and used for Real-Time PCR for Type I collagen (FIG. 1C) or Type III collagen (FIG. 1D). Data represents n=2 for ets-2 (WT/WT)+PBS; n=5 for ets-2 (A72/A72)+PBS; n=5 for ets-2 (WT/WT)+bleomycin; n=7 for ets-2 (A72/A72)+bleomycin.

FIGS. 2A-2D: ets-2 (A72/A72) mice exhibit decreased CCL3 and CCL12 expression following bleomycin treatment when compared to ets-2 (WT/WT) mice. Ets-2 (WT/WT) and ets-2 (A72/A72) mice were treated with bleomycin (0.035 U/g) or PBS (vehicle control). RNA from mouse lungs was isolated, and mRNA was amplified and used for Real-Time PCR for CCL3 (FIG. 2A) or CCL12 (FIG. 2B). Data represents n=6 for ets-2 (WT/WT)+bleomycin; n=7 for ets-2 (A72/A72)+bleomycin.

FIGS. 2C-2D: Following sacrifice, a bronchoalveolar lavage (BAL) was performed with 1.5 ml of PBS. The resulting BAL fluid was analyzed for levels of CCL12 (FIG. 2C) and CCL2 (FIG. 2D) via ELISA. Data represents n=5 for ets-2 (WT/WT)+PBS; n=6 for ets-2 (A72/A72)+PBS; n=8 for ets-2 (WT/WT)+bleomycin; n=9 for ets-2 (A72/A72)+bleomycin.

FIGS. 3A-3F: Lungs from ets-2 (A72/A72) mice have reduced expression of active TGFβ, αSMA, type-I collagen, and CTGF following bleomycin administration. Ets-2 (WT/WT) and ets-2 (A72/A72) mice were treated with bleomycin (0.035 U/g) or PBS (vehicle control).

FIG. 3A: Following sacrifice, BAL fluid was analyzed for active TGFβ via ELISA. Data represents n=3 for ets-2 (WT/WT)+PBS; n=4 for ets-2 (A72/A72)+PBS; n=6 for ets-2 (WT/WT)+bleomycin; n=8 for ets-2 (A72/A72)+bleomycin.

FIGS. 3B-3E: Lungs from bleomycin treated mice underwent immunohistochemical staining for α-smooth muscle actin (SMA) (FIG. 3B) or type I collagen (FIG. 3C). Quantification of this is shown in FIG. 3D and FIG. 3E, respectively. Data is representative of at least 3 mice per condition.

FIG. 3F: RNA from mouse lungs was also isolated, and mRNA was amplified and used for Real-Time PCR for CTGF (black bars) or αSMA (gray bars). Data represents n=2 for ets-2 (WT/WT)+PBS; n=5 for ets-2 (A72/A72)+PBS; n=5 for ets-2 (WT/WT)+bleomycin; n=7 for ets-2 (A72/A72)+bleomycin.

FIGS. 4A-4B: Lung sections from patients with idiopathic pulmonary fibrosis (IPF) have increased levels of phosphorylated ets-2 that co-localizes with type-I collagen expression.

FIG. 4A: Human IPF lung tissue: phospho-ets-2 IHC, red stain. Additional dual staining with type-I collagen.

FIG. 4B: Type-I collagen alone on human IPF lung tissue, red stain. Brown staining a background marker.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention is based, at least in part, on the inventors' discovery that the ets-2 (A72/A72) mice are protected from bleomycin-induced pulmonary fibrosis compared to ets-2 (WT/WT) mice. This protection did not correlate to changes in inflammatory cell recruitment to the lung or inflammatory cytokine gene expression in the lung. However, the protection of the ets-2 (A72/A72) mice correlated with decreased fibrotic gene expression, including reduced CCL12, connective tissue growth factor (CTGF), type-I collagen, and α-smooth muscle actin (αSMA), as evidenced by real time PCR, ELISA analysis, and immunohistochemical staining.

Furthermore, lung samples from patients with IPF exhibited a marked increase in ets-2 activation, as evidenced by immunohistochemical methods, with the staining occurring along the leading fibrotic edge in pneumocytes and myofibroblasts. In addition, the activated ets-2-positive myofibroblasts along the leading fibrotic edge also stained positive for type-I collagen, highlighting the important roles of ets-2 in the pathogenesis of pulmonary fibrosis in mice and patients with IPF.

The inventors have now determined the role of the transcription factor ets-2 in the pathogenesis of pulmonary fibrosis. While the inventors do not intend to be bound by theory, the following is a description of the basis for the invention.

The transcription factor ets-2 becomes activated following phosphorylation at threonine-72. Transgenic mice containing a single amino acid mutation at threonine-72 to alanine [ets-2 (A72/A72)] exhibit a dramatic reduction in basal lung inflammation when crossed with the moth-eaten viable [hcph (me-v/me-v)] mouse.

In a general aspect, the methods followed by the inventors were as follows: Following intra-peritoneal injection of bleomycin into mice, pulmonary fibrosis was measured by pathological assessment, quantification of lung collagen (Sircol Assay), bronchoalveolar lavage analysis of inflammatory cytokines (ELISA) and cell differentials, real time PCR for mRNA expression profiling, and immunohistochemical staining. Lung sections from patients with idiopathic pulmonary fibrosis (IPF) were analyzed for activation of ets-2 by immunohistochemical methods.

It is now shown herein that the ets-2 (A72/A72) mice were protected from bleomycin-induced pulmonary fibrosis, as evidenced by decreased subpleural collagen deposition and reduced interstitial thickening of the lungs when compared to ets-2 (WT/WT) mice. Although the inflammatory cell profile from the lungs of ets-2 (wt/wt) and ets-2 (A72/A72) mice did not differ following bleomycin, the ets-2 (A72/A72) mice demonstrated a significant decrease in the expression of several important fibrotic factors, including MCP-5/CCL12, active TGFβ, type-I collagen, connective tissue growth factor (CTGF), and α-smooth muscle (αSMA) actin, as evidenced by real time PCR, ELISA analysis, and immunohistochemical staining.

Also, lung sections from patients with IPF contained prominent areas of ets-2 phosphorylation (at threonine-72) that co-localized with type-I collagen staining when compared to normal lung sections from patients without IPF. The main areas of positive staining occurred along the leading fibrotic edges and were localized to pneumocytes and myofibroblasts.

The inventors herein now show the importance of ets-2 in directing the fibrotic pathways involved in the pathogenesis of pulmonary fibrosis and, particular, in patients with IPF.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified. The value of the present invention can thus be seen by reference to the Examples herein.

### Examples

### Methods

Mice: Transgenic mice harboring a point mutation in Ets2 [Ets2tmA720sh, referred to as Ets2 (A72/A72)] resulting in a threonine to alanine conversion at position 72 were used. Wild type littermates (FVB/N background) were used as controls.

Bleomycin studies: Male mice, aged 6-12 weeks, underwent intra-peritoneal (IP) injection. Briefly, mice were injected with 0.035 U bleomycin/g or PBS (vehicle control) on days 1, 4, 8, 11, 15, 18, 22, and 25. One week following the last injection, mice were sacrificed. Prior to removal, lungs were lavaged with 1.0 ml of PBS. Lungs were subsequently removed and inflated at 20 cm pressure. The left lobe of the lung was placed in 10% Formalin and prepared for immunohistochemical processing (Histotechniques, Powell, Ohio), and the right lobes were snap frozen in liquid nitrogen for RNA, collagen, and protein analyses.

Analysis of Lung Fibrosis:

Real Time PCR: Total RNA was extracted from lung tissue by freeze fracture and Trizol extractions. Reverse Transcriptase was done to amplify only mRNA by using oligo-dT primers (Invitrogen). cDNA was analyzed and used for Real Time PCR with SYBR Green. All primers were designed using Primer Express software (Applied Biosystems).

Primer sequences:
mouse GAPDH:
5′-GCACAGTCAAGGCCGAGAAT-3′; (For) [SEQ ID NO: 1]
5′-GCCTTCTCCATGGTGGTGAA-3′; (Rev) [SEQ ID NO: 2]
mouse type I Collagen:
5′-ATGGATTCCCGTTCGAGTACG-3′; (For) [SEQ ID NO: 3]
5′-TCAGCTGGATAGCGACATCG-3′; (Rev) [SEQ ID NO: 4]
mouse type III Collagen:
5′-CACCCTTCTTCATCCCACTCTTA-3′; (For) [SEQ ID NO: 5]
5′-ACCAAGGTGGCTGCATCC-3′; (Rev) [SEQ ID NO: 6]
mouse CCL12:
5′-TGGCTGGACCAGATGCG-3′; (For) [SEQ ID NO: 7]
5′-GACGTGAATCTTCTGCTTAACAACA-3′; (Rev)[SEQ ID NO: 8]
mouse tenascin-C:
5′-TGTGTGCTTCGAAGGCTATG-3′; (For) [SEQ ID NO: 9]
5′-GCAGACACACTCGTTCTCCA-3′; (Rev) [SEQ ID NO: 10]
mouse α-smooth muscle actin:
5′-CTGACAGAGGCACCACTGAA-3′; (For) [SEQ ID NO: 11]
5′-CATCTCCAGAGTCCAGCACA-3′; (Rev) [SEQ ID NO: 12]
mouse CTGF:
5′-AAAGTGCATCCGGACACCTAA-3′; (For) [SEQ ID NO: 13]
5′-TGCAGCCAGAAAGCTCAAACT-3′. (Rev) [SEQ ID NO: 14]

Real Time PCR analysis was done using an ABI 7700 machine (Applied Biosystems) with GAPDH as an internal control.

Bronchoalveolar lavage (BAL) analysis: Following the bleomycin regimen (33 days), two instillations of 0.5 ml of PBS were performed, and the BAL fluid (BALF) was collected. The total number of cells isolated via BAL was calculated. BAL fluid was then centrifuged to pellet the cells, and the supernatant (BALF) was collected and stored for ELISA analyses. Cells were resuspended in 100 μl of PBS, cytospun on slides, stained, and analyzed under a microscope for cell differentials.

Sircol Collagen Assay: Mouse lungs were homogenized in 1.5 ml of 0.5 M acetic acid and rocked overnight at 4° C. The samples were centrifuged for 10 minutes at 2000 rpm, and the resulting supernatant was assessed for collagen according to the manufacturer's instructions.

ELISA analysis: Analysis was done using an ELx808 Microplate Reader (Bio-TEK Instruments, Winooski, Vt.). A standard curve r2 value of no less than 0.98 was considered acceptable for sample analysis. For all ELISAs, the protocol given by the manufacturer (R&D Systems, Inc) was followed.

Immuno-histochemical staining and quantification: All immuno-histochemical staining of mouse lung sections was performed by the Pathology Core Facility at The Ohio State University College of Medicine (Columbus, Ohio). α-Smooth muscle actin and type-I collagen expression in the lungs were quantified using histogram analysis in Adobe Photoshop CS2 by converting all SMA(+)-brown stained or all collagen I(+)-brown stained pixels to black and all other lung tissue to white. The two stains were analyzed on separate slides for the same lung. The values indicated represent the % of SMA(+) or collagen I(+) cells per high-powered field from at least 10 digital images per lung per mouse. Immuno-histochemical staining for phosphorylated human ets-2 and type-I collagen on human lung tissues from patients with IPF or patients without IPF was performed using a Ventana Medical Systems automated immunohistochemistry Benchmark according to the manufacturer's recommendations. The antibody for phosphorylated ets-2 (targeted for threonine-720) was used at a 1:10 dilution using an antigen retrieval method for lung sections. The antibody for human type-I collagen was purchased from Abcam (Cambridge, Mass.) and was used at a 1:750 dilution.

Statistics: A student's T-test was used for single comparisons, with a p-value<0.05 considered significant. ANOVA was used for multiple comparisons. ANOVA requires that the cell counts be normally distributed with stable variance across groups. Thus the cell counts were log transformed in order to meet these assumptions. If the overall ANOVA F-test is significant, indicating that differences exist in the data, then individual groups were tested to determine if these differences were statistically significant. The p-values were adjusted using the Holm's procedure to control the type I error at 5%. All analyses were run using Stata 10.0, Stata Corporation, College Station, Tex. Data was considered significant with a p-value≦0.05.

Results

Ets-2 (A72/A72) Mice are Protected from Bleomycin-Induced Pulmonary Fibrosis.

M-CSF is of importance in the pathogenesis of pulmonary fibrosis. M-CSF is a hematopoietic growth factor that can activate the transcription factor ets-2 in macrophages, with phosphorylation occurring at threonine-72. Cross-breeding and hcph (me-v/me-v) mice with mutant ets-2 (A72/A72) transgenic mice dramatically reduces lung inflammation and improves survival. Since the moth-eaten-viable mouse (me-v/me-v) is a naturally occurring model of interstitial lung disease, the inventors herein examined the role of ets-2 and the ets-2 (A72/A72) mice in bleomycin-induced pulmonary fibrosis.

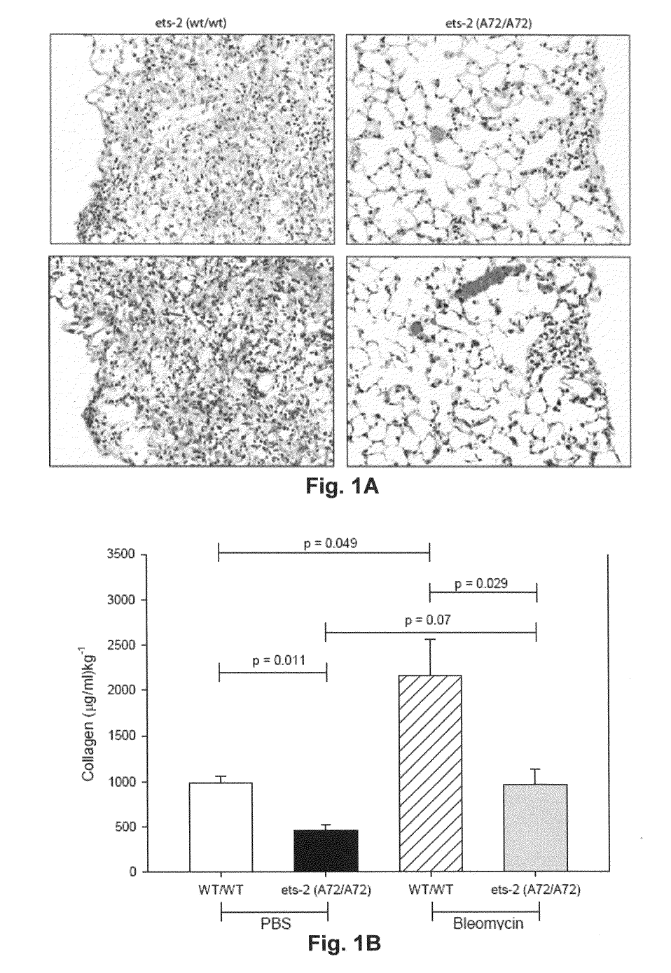

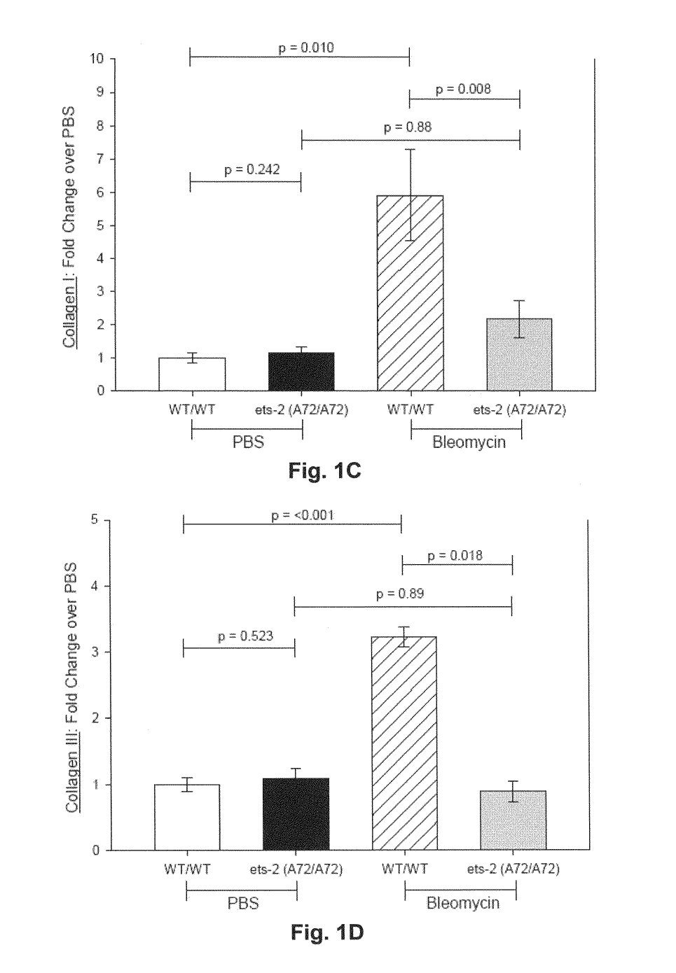

As shown in FIG. 1A, ets-2 (A72/A72) mice had reduced lung injury and subpleural collagen deposition following bleomycin administration compared to ets-2 (WT/WT) mice. While ets-2 (A72/A72) mice had small amounts of interstitial thickening and fibrosis in the sub-pleural region of the lung, there was significantly more interstitial thickening and fibrosis in ets-2 (WT/WT) mice treated with bleomycin.

The amount of lung collagen was assessed following bleomycin challenge using the Sircol Collagen assay. The ets-2 (A72/A72) mice had significantly less collagen in their lungs compared to ets-2 (WT/WT) mice (FIG. 1B).

Further analysis revealed that ets-2 (A72/A72) mice expressed significantly less type I and III collagen mRNA in the lungs following bleomycin treatment when compared to bleomycin-treated ets-2 (WT/WT) mice, as assessed by real time PCR (FIG. 1C and FIG. 1D, respectively). These data are unexpected and show the importance of mutant ets-2 in protecting mice from bleomycin-induced lung injury.

Ets-2 (A72/A72) Mice Exhibit Reduced CCL12 Expression Following Bleomycin Administration.

With the ets-2 (A72/A72) mice, the expression of CCL3 in the lungs is significantly decreased in the [ets-2 (A72/A72)/hcph (me-v/me-v)] mice compared to [ets-2 (WT/WT)/hcph (me-v/me-v)] mice (17), indicating that CCL3 is an ets-2-dependent gene. CCL3 is linked to the pathogenesis of pulmonary fibrosis in mice and humans, and the inventors herein now believe that CCL3 expression in the lung differs between ets-2 (A72/A72) and ets-2 (WT/WT) mice following bleomycin treatment.

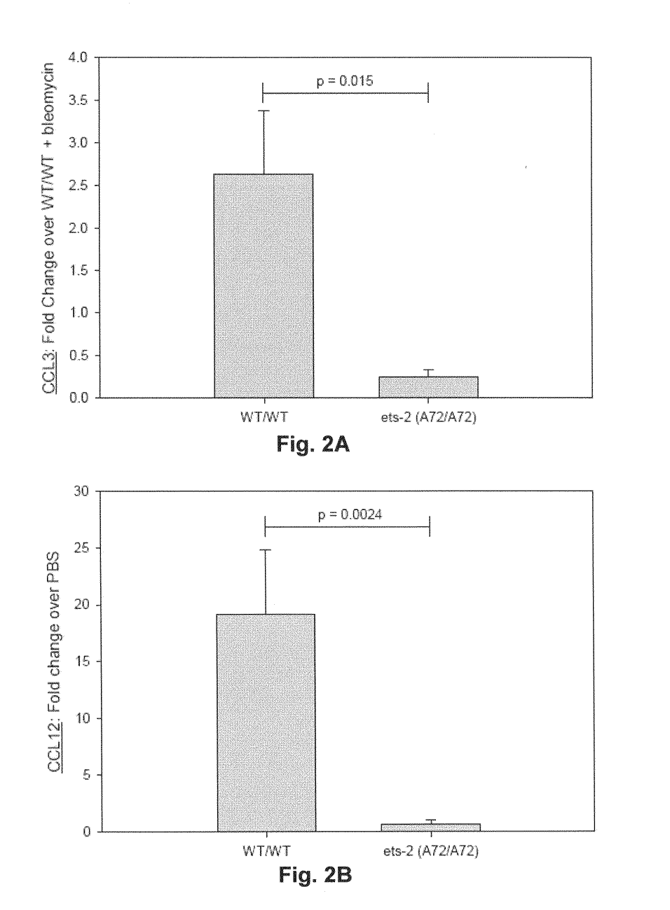

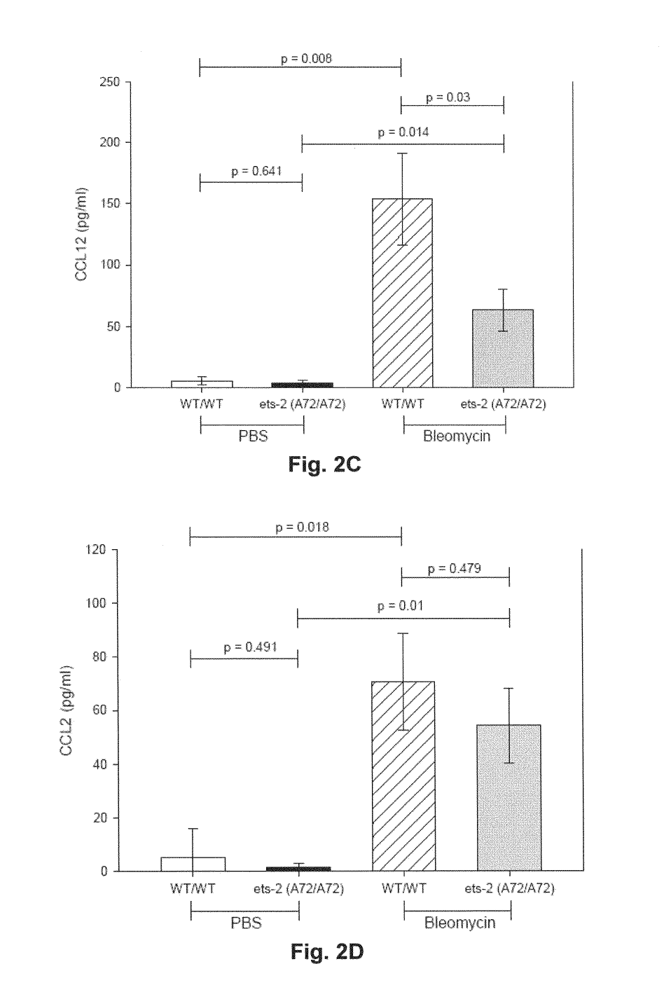

The inventors have now discovered that ets-2 (A72/A72) mice expressed significantly less CCL3 mRNA in the lungs following bleomycin treatment, as compared to ets-2 (WT/WT) mice (FIG. 2A), indicating the specificity of ets-2 in regulating CCL3 expression.

To determine if CCL3 was responsible for protection seen in bleomycin-challenged ets-2 (A72/A72) mice, the inventors obtained CCL3 (−/−) mice and challenged them or background-matched CCL3 (+/+) mice with intra-peritoneal bleomycin. The inventors herein have now unexpectedly shown that CCL3 (−/−) mice were not protected from intra-peritoneal bleomycin administration compared to CCL3 (+/+) mice (data not shown).

The value of the present invention is further evidenced by the showing that, although ets-2 directs the expression of CCL3 and is increased in the lungs of bleomycin-treated mice, this cytokine did not appear to be the ets-2-dependent factor responsible for determining fibrosis in bleomycin-challenged mice.

Since CCL12 as an important mediator of pulmonary fibrosis in response to bleomycin in vivo and M-CSF in vitro, the inventors herein assessed the levels of CCL12 mRNA in the lungs of ets-2 (WT/WT) and ets-2 (A72/A72) mice after bleomycin treatment.

FIG. 2B demonstrates that ets-2 (A72/A72) mice, following bleomycin treatment, expressed significantly less CCL12 mRNA, as compared to ets-2 (WT/WT) mice. The inventors also evaluated expression levels of CXCL12, but observed no statistical difference in whole lung mRNA expression between ets-2 (WT/WT) and ets-2 (A72/A72) after bleomycin administration (data not shown).

To verify that mRNA levels correlated with protein expression, the inventors also analyzed bronchoalveolar (BAL) fluid from these mice. ELISA analysis revealed that ets-2 (A72/A72) mice expressed significantly less CCL12 protein in BAL fluid when compared to ets-2 (WT/WT) mice following bleomycin treatment (FIG. 2C).

Unexpectedly, there was no significant difference in CCL2 expression between ets-2 (WT/WT) and ets-2 (A72/A72) after bleomycin treatment (FIG. 2D).

To further characterize the protection from bleomycin-induced pulmonary fibrosis offered to ets-2 (A72/A72) mice, the inventors analyzed the inflammatory cell profile in the lung following bleomycin treatment. As shown in Table 1, BAL fluid analysis revealed no significant difference in the number of macrophages, lymphocytes, or neutrophils recruited to the lungs of ets-2 (A72/A72) and ets-2 (wt/wt) mice following bleomycin.

TABLE 1
Bronchoalveolar Lavage (BAL) Cell Differentials

| | | | Alveolar | | |
|---|---|---|---|---|---|
| | | Total cells | Macrophages | Neutrophils | Lymphocytes |
| ets-2 genotype | Treatment | ×10⁴ | ×10⁴ | ×10⁴ | ×10⁴ |
| (WT/WT) | PBS | 2.9 ± 0.6 | 2.9 ± 0.6 | 0 | 0 |
| n = 3 | | | | | |
| (WT/WT) | Bleomycin | 12.0 ± 2.3*, # | 12.0 ± 2.3†, $ | 0 | 0 |
| n = 5 | | | | | |
| (A72/A72) | PBS | 2.6 ± 0.5 | 2.6 ± 0.5 | 0 | 0 |
| n = 4 | | | | | |
| (A72/A72) | Bleomycin | 8.3 ± 2.0** | 6.8 ± 1.4†† | 0.8 ± 0.6 | 0.7 ± 0.6 |
| n = 7 | | | | | |

Data represents mean ± S.E.M.
*p = 0.0155 vs (WT/WT) PBS;
**p = 0.1304 vs (A72/A72) PBS;
†p = 0.0085 vs (WT/WT) PBS;
††p = 0.1705 vs (A72/A72) PBS;
#p = 0.3714 vs (A72/A72) Bleomycin;
$p = 0.1622 vs (A72/A72) Bleomycin.

The number of mononuclear phagocytes in the lungs of bleomycin-treated ets-2 (A72/A72) and ets-2 (wt/wt) mice likely reflects similar CCL2 levels between the two mice (FIG. 2D). Taken together, these data show that the ets-2 (A72/A72) mice do not have a significant difference in the inflammatory profile following bleomycin treatment when compared to ets-2 (WT/WT) mice, but do exhibit reduced expression of the fibrotic factors CCL12 and type I and type III collagen.

Lungs from ets-2 (A72/A72) Mice Exhibit Reduced Pro Fibrotic Gene Expression in Response to Bleomycin.

Since the ets-2 (A72/A72) mice express less CCL12 and type I and III collagen in the lungs following bleomycin treatment, the inventors now believe that other fibrotic factors are differentially expressed in the lungs of ets-2 (A72/A72) mice following bleomycin treatment.

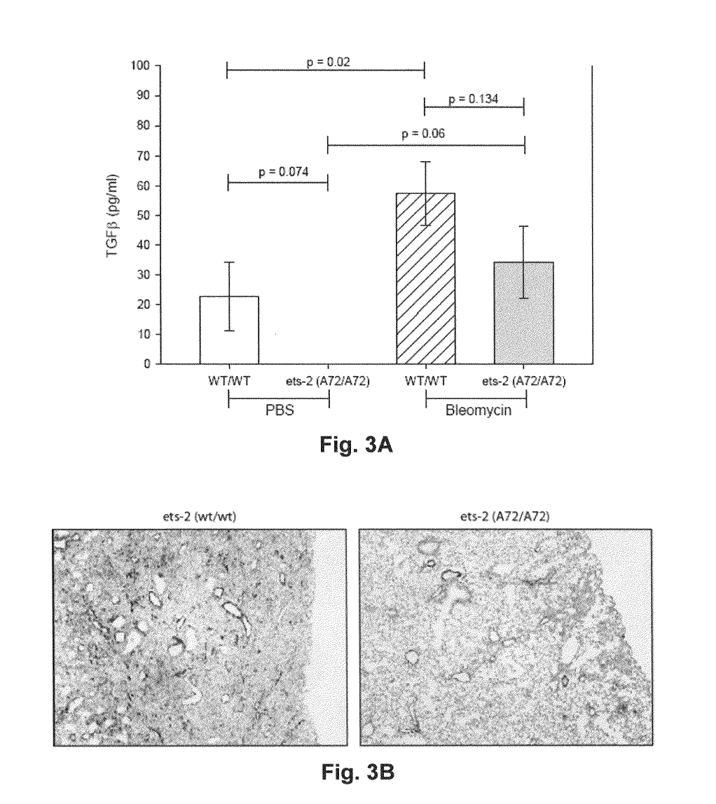

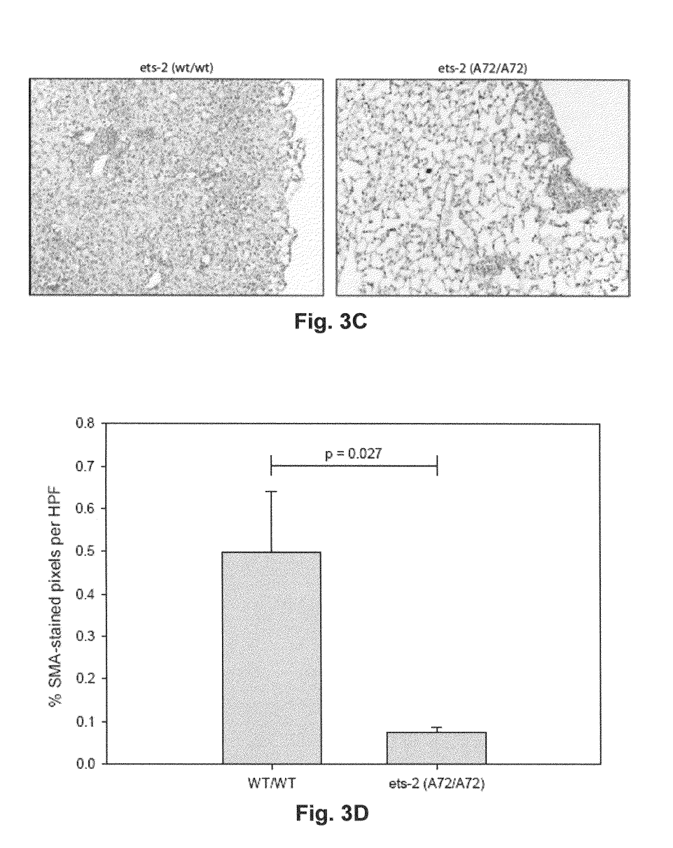

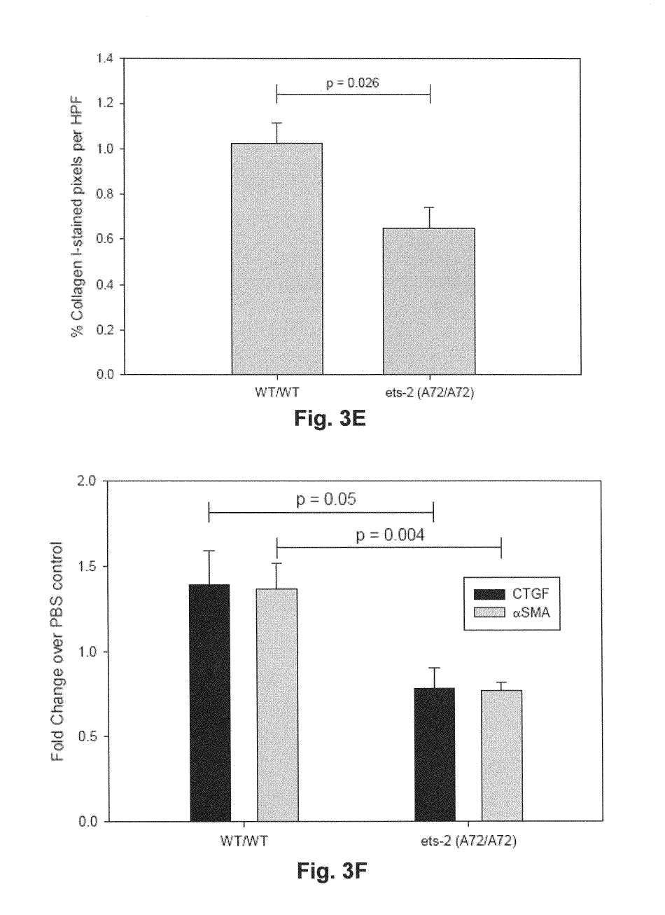

One of the hallmark proteins in murine models of pulmonary fibrosis and in patients with IPF is active TGFβ. Analysis of the BAL fluid from ets-2 (A72/A72) and ets-2 (WT/WT) mice revealed a significant decrease in the amount of active TGFβ following bleomycin treatment in ets-2 (A72/A72) mice compared to ets-2 (WT/WT) mice (FIG. 3A).

Similar to active TGFβ, myofibroblasts have also been well established as important mediators of fibrosis in response to bleomycin and in patients with IPF. These cells are characterized by the expression of various factors, including α-smooth muscle actin (αSMA) and collagen. Bleomycin-treated ets-2 (A72/A72) mouse lungs contained significantly fewer αSMA-positive cells and significantly fewer type-I collagen positive cells, as compared to ets-2 (WT/WT), assessed by immunohistochemical staining (FIG. 3B and FIG. 3C). Quantification of this data is shown in FIG. 3D and FIG. 3E, respectively.

Lungs from ets-2 (A72/A72) mice have decreased expression of αSMA mRNA (FIG. 3F) and type-I collagen mRNA (as shown in FIG. 1C) following bleomycin treatment compared to ets-2 (WT/WT) mice.

The ets-2 (A72/A72) mice also expressed less CTGF mRNA following bleomycin treatment compared to ets-2 (WT/WT) mice (FIG. 3F). CTGF is an important fibrotic growth factor in numerous models of organ fibrosis, including the lung, and is directly involved in the expression of αSMA and collagen. Interestingly, expression of the pro-fibrotic factor tenascin-C, which is involved in pulmonary fibrotic disease and contains a requisite ets-2 binding site in its promoter, is not differentially expressed between the ets-2 (WT/WT) and ets-2 (A72/A72) mice after bleomycin treatment (data not shown). Similarly, expression of CCR2, the receptor that binds CCL2 and CCL12 that directs a fibrotic cytokine cascade and collagen deposition in fibroblasts, is also not differentially expressed between ets-2 (WT/WT) and ets-2 (A72/A72) mice (data not shown).

Although the BAL inflammatory cell differential was not statistically different between ets-2 (A72/A72) and ets-2 (WT/WT) mice, a significant number of pro-fibrotic factors that govern fibrotic lung disease, including CCL12, CTGF, active TGFβ, αSMA, and type-I collagen, are all significantly reduced in the ets-2 (A72/A72) mice following bleomycin treatment.

Lung Sections from Patients with Idiopathic Pulmonary Fibrosis (IPF) have Increased Levels of Phosphorylated ets-2.

To translate the findings from the mouse model of bleomycin-induced pulmonary fibrosis to human lung disease, the inventors determined the expression pattern of phosphorylated ets-2 in lung sections from patients with IPF. The transcription factor ets-2 becomes phosphorylated at threonine-72, accumulates in the nucleus, and results in the mediation of downstream signaling events and gene transcription. Therefore, to assess the levels of ets-2 phosphorylation in human lung sections, the inventors utilized an antibody that specifically targets phosphorylated human ets-2 at threonine-72 via immunohistochemistry.

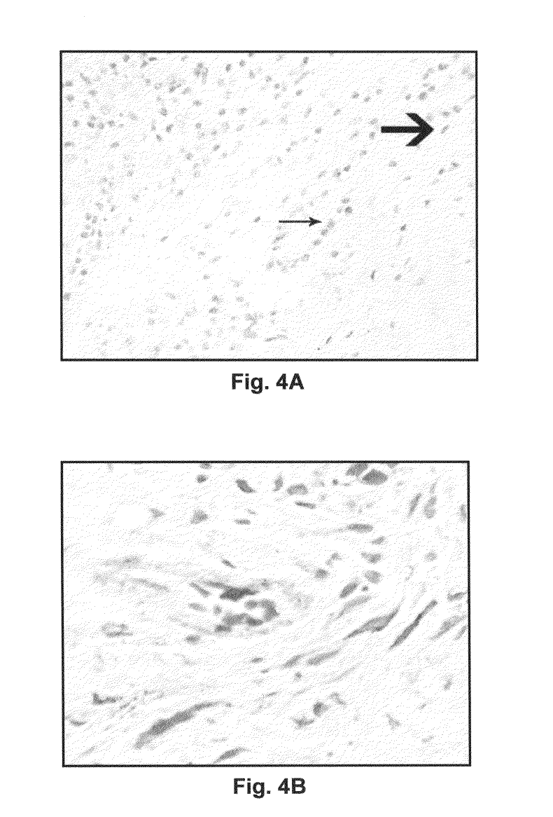

As shown in FIG. 4A, the pattern of phosphorylated ets-2 in normal lung sections was absent, while lung sections from patients with IPF exhibited pronounced levels of phosphorylated ets-2 along the leading fibrotic edge, with pneumocytes and myofibroblasts being the primary cellular source. Lung sections were also stained with type-I collagen Type-I collagen dual IHC data.

### Discussion

Ets-2 plays an important causal role in bleomycin-induced pulmonary fibrosis. Because CCL3 is an ets-2-dependent gene and is implicated in causing bleomycin-induced pulmonary fibrosis, the inventors assayed CCL3 expression in ets-2 (A72/A72) mice. CCL3 expression was reduced in the lungs of ets-2 (A72/A72) mice after bleomycin treatment. However, CCL3 (−/−) mice were not protected against pulmonary fibrosis in response to intra-peritoneal bleomycin. This discovery is in contrast to previous studies demonstrating that CCL3 is critical in the pathogenesis of a single dose, intra-tracheal model of pulmonary fibrosis using CCL3 (−/−) mice. There are several key differences between these studies, including the route of administration (intra-tracheal versus intra-peritoneal) and the number of bleomycin injections (one versus eight, respectively). In fact, the inventors recently found that the model of repeat intra-peritoneal bleomycin injections in this murine model of pulmonary targets different fibrotic protein pathways than intra-tracheally injected mice. For example, the inventors found that CCL2 (−/−) mice had less lung fibrosis after intra-peritoneal bleomycin, whereas a separate publication that used the intra-tracheal injection model demonstrated no protection from lung fibrosis in these mice. Although the inventors do not discount the potential importance of CCL3 in lung inflammation, the inventors' data did not support CCL3 as the ets-2-dependent factor responsible for intra-peritoneal bleomycin-induced pulmonary fibrosis.

Since murine CCL2 expression in the lungs of bleomycin-treated ets-2 (A72/A72) and ets-2 (wt/wt) mice was not statistically different, the inventors looked for alternative ets-2-dependent proteins that may be responsible. The inventors discovered that the expression of CCL12 and α-SMA were markedly different in the lungs of ets-2 (A72/A72) mice, as compared to ets-2 (WT/WT) mice after intra-peritoneal bleomycin injection. CCL12 is important in fibrocyte recruitment to the lung and uses the CCR2 receptor to target these cells. Finding that CCL12 is regulated by ets-2 provides valuable insight into the molecular regulation of fibrocyte recruitment and myofibroblast expansion within the lung.

Interestingly, the human homolog to murine CCL12 is CCL2. Initial analysis of the human CCL2 promoter did not reveal a functional ets-2 binding site. In addition to connecting ets-2 and CCL12, the inventors now believe that additional ets-2 dependent cytokines or chemokines are may facilitate fibrocyte recruitment to the lung and myofibroblast expansion within the lung.

The inventors herein also now show that a single amino acid mutation in ets-2 (threonine-72 to alanine) protected mice from bleomycin-induced pulmonary fibrosis by reducing fibrotic gene expression in the lung.

The inventors herein now show that patients with IPF have dramatically increased levels of phosphorylated ets-2 in the lung along the leading fibrotic edge.

The inventors herein now show that mice expressing a mutant form of ets-2 were protected against intra-peritoneal injections of bleomycin, had lower expression of CCL12 in the lungs, and had less collagen type I and α-smooth muscle actin-positive cells in the lungs by immunostaining.

Examples of Uses

Modulating Expression

The step of modulating the expression of the transcription factor can be accomplished in many ways that are known to one of ordinary skill in the art, one non-limiting example includes inhibiting the activation of the promoter for the gene encoding the transcription factor. In certain embodiments, the step of inhibiting activation further comprises providing a substance that blocks the function or expression of the transcription factor. The substance can be selected by one of ordinary skill in the art but can include, for example, small molecules, peptides, dominant negative mutants, antisense RNAs, and DNA viruses.

The methods in which the activity of the transcription factor is increased can be accomplished in many ways that are known to one of ordinary skill in the art, e.g., activating the promoter for the gene encoding the transcription factor. In certain embodiments, the step of increasing activation further comprises providing a substance that increases the function or expression of the transcription factor. The substances can be selected by one of ordinary skill in the art but include, small molecules, peptides, dominant positive mutants, antisense RNAs, and DNA viruses. Preferred substances mimic or enhance the activity of the transcription factor.

The substance that alters the activity of the transcription factor can be provided in vivo systemically, or alternatively, the substance is provided to the site of fibrosis, depending on the result desired. For example, the substance (e.g., small molecule drugs, peptides, dominant negative mutants by gene delivery mechanisms, antisense RNA) can be used to block the function or expression of the transcription factor ets-2, systemically to treat a fibrosis-related disease such as idiopathic pulmonary fibrosis (IPF). Alternatively, local delivery of an ets-2 blocking agent can be used to treat localized fibrosis. Methods of in vivo administration, such as gene therapy are known to one of ordinary skill in the art.

Screening

The invention provides methods of screening compounds that are capable of reducing fibrosis. One such method comprises: (a) providing cells which do not normally express a measurable transcription factor but do express the transcription factor in the presence of a pro-fibrosis agent; (b) providing to a portion of the cells a compound to be screened; (c) providing a portion of the cells as a control without the compound; (d) providing the pro-fibrosis agent to the cells; (e) measuring the expression of the transcription factor in the cells, and (f) comparing the amount of expression of the transcription factor in the cells containing the compound with the control portion of cells. In preferred methods, the transcription factor is an ets transcription factor. In especially preferred methods, the transcription factor is ets-2. Examples of cells which are useful in screening methods of the present invention include, but are not limited to, myofibroblasts.

The invention further provides screening methods where the compound of interest is a small molecule, peptide, antisense RNA or viral DNA. The fibrosis agent can be selected by one of ordinary skill in the art.

Agonists and Antagonists

In accordance with another aspect of the present invention, there are provided ets-2 agonists. Among preferred agonists are molecules that mimic ets-2, that bind to ets-2-binding molecules or receptor molecules, and/or that elicit or augment ets-2-induced responses. Also among preferred agonists are molecules that interact with ets-2 or ets-2 polypeptides, or with other modulators of ets-2 activities, and thereby potentiate or augment an effect of ets-2 or more than one effect of ets-2.

In accordance with yet another aspect of the present invention, there are provided ets-2 antagonists. Among preferred antagonists are those which mimic ets-2 so as to bind to an ets-2 receptor or binding molecules, but not elicit an ets-2-induced response or more than one ets-2-induced response. Also among preferred antagonists are molecules that bind to and/or interact with ets-2 so as to inhibit an effect of ets-2 or more than one effect of ets-2 or which prevents expression of ets-2.

The invention also provides methods of diagnosing the presence of an fibrotic disease in a subject comprising: i) removing a sample from the subject, and ii) measuring the presence and/or amount of transcription factor ets-2 wherein the transcription factor ets-2 is not present in detectable amounts in the sample in the absence of the fibrotic disease. In the methods of the present invention, the sample comprises tissue or BALF.

The invention further provides a method of monitoring the treatment of a fibrotic disease in a subject comprising: removing a sample from the subject subsequent to a treatment for IPF and measuring the presence or amount of transcription factor ets-2, wherein the transcription factor is not present in detectable amounts in the subject in the absence of the disease.

In certain embodiments of these methods, the method further comprises repeating the removing and measuring steps at subsequent intervals and comparing the amounts of the transcription factor to determine if the treatment is effective.

Compositions

The present invention also relates to a pharmaceutical composition for the treatment of fibrotic-related diseases comprising a compound that alters the expression of the transcription factor ets-2, and a pharmaceutically acceptable carrier. Preferred compositions comprise compounds that alter the expression of ets-2 in Lung tissue. Examples of compounds that are useful in such compositions include small molecules, peptides, or antisense RNA.

The present invention further relates to altering the expression of a fibrosis response gene comprising modulating the expression of a transcription factor which affects the expression of the fibrosis response gene. In some embodiments, the step of altering the expression of the fibrosis response gene comprises decreasing the expression or the activity of the transcription factor. The step of decreasing the activity of the transcription factor can further comprise either decreasing the function of the transcription factor or blocking the expression of the transcription factor. In other embodiments, altering the expression of the fibrosis response gene comprises increasing the activity of the transcription factor. In certain of these embodiments, the step of increasing the activity of a transcription factor can further comprise either increasing the function of the transcription factor or increasing the expression of the transcription factor.

The present invention further relates to a method of treating a disease comprising increasing the activity of a transcription factor, wherein the transcription factor is either not expressed in diseased tissue or expressed in low amounts. The transcription factor increases the expression of a product that is useful for treating the disease.

Kits and Arrays

Any of the compositions described herein may be comprised in a kit. In a non-limiting example, reagents for isolating RNA, labeling RNA, and/or evaluating an RNA population using an array are included in a kit. The kit may further include reagents for creating or synthesizing RNA probes. The kits will thus comprise, in suitable container means, an enzyme for labeling the RNA by incorporating labeled nucleotide or unlabeled nucleotides that are subsequently labeled. It may also include one or more buffers, such as reaction buffer, labeling buffer, washing buffer, or a hybridization buffer, compounds for preparing the RNA probes, and components for isolating RNA. Other kits may include components for making a nucleic acid array comprising oligonucleotides complementary to RNAs, and thus, may include, for example, a solid support.

For any kit embodiment, including an array, there can be nucleic acid molecules that contain a sequence that is identical or complementary to all or part of any of sequences described herein.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being one preferred solution. Other solutions that may be included in a kit are those solutions involved in isolating and/or enriching RNA from a mixed sample.

However, the components of the kit may be provided as dried powder(s). When reagents and/or components are provided as a dry powder, the powder can be reconstituted by the addition of a suitable solvent. It is envisioned that the solvent may also be provided in another container means. The kits may also include components that facilitate isolation of the labeled RNA. It may also include components that preserve or maintain the RNA or that protect against its degradation. The components may be RNAse-free or protect against RNAses.

Also, the kits can generally comprise, in suitable means, distinct containers for each individual reagent or solution. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of RNA.

It is also contemplated that any embodiment discussed in the context of an RNA array may be employed more generally in screening or profiling methods or kits of the invention. In other words, any embodiments describing what may be included in a particular array can be practiced in the context of RNA profiling more generally and need not involve an array per se.

It is also contemplated that any kit, array or other detection technique or tool, or any method can involve profiling for any of these RNAs. Also, it is contemplated that any embodiment discussed in the context of an RNA array can be implemented with or without the array format in methods of the invention; in other words, any RNA in an RNA array may be screened or evaluated in any method of the invention according to any techniques known to those of skill in the art. The array format is not required for the screening and diagnostic methods to be implemented.

The kits for using RNA arrays for therapeutic, prognostic, or diagnostic applications and such uses are contemplated by the inventors herein. The kits can include an RNA array, as well as information regarding a standard or normalized RNA profile for the RNAs on the array. Also, in certain embodiments, control RNA or DNA can be included in the kit. The control RNA can be RNA that can be used as a positive control for labeling and/or array analysis.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

Array Preparation and Screening

Also provided herein are the preparation and use of RNA arrays, which are ordered macroarrays or microarrays of nucleic acid molecules (probes) that are fully or nearly complementary or identical to a plurality of RNA molecules or precursor RNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of RNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass and silicon. The arrays may vary in a number of different ways, including average probe length, sequence or types of probes, nature of bond between the probe and the array surface, e.g. covalent or non-covalent, and the like. The labeling and screening methods described herein and the arrays are not limited in its utility with respect to any parameter except that the probes detect RNA; consequently, methods and compositions may be used with a variety of different types of RNA arrays.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

mouse GAPDH:
5′-GCACAGTCAAGGCCGAGAAT-3′; (For) [SEQ ID NO: 1]
5′-GCCTTCTCCATGGTGGTGAA-3′; (Rev) [SEQ ID NO: 2]
mouse type I Collagen:
5′-ATGGATTCCCGTTCGAGTACG-3′; (For) [SEQ ID NO: 3]
5′-TCAGCTGGATAGCGACATCG-3′; (Rev) [SEQ ID NO: 4]
mouse type III Collagen:
5′-CACCCTTCTTCATCCCACTCTTA-3′; (For) [SEQ ID NO: 5]
5′-ACCAAGGTGGCTGCATCC-3′; (Rev) [SEQ ID NO: 6]
mouse CCL12:
5′-TGGCTGGACCAGATGCG-3′; (For) [SEQ ID NO: 7]
5′-GACGTGAATCTTCTGCTTAACAACA-3′; (Rev) [SEQ ID NO: 8]
mouse tenascin-C:
5′-TGTGTGCTTCGAAGGCTATG-3′; (For) [SEQ ID NO: 9]
5′-GCAGACACACTCGTTCTCCA-3′; (Rev) [SEQ ID NO: 10]
mouse α-smooth muscle actin:
5′-CTGACAGAGGCACCACTGAA-3′; (For) [SEQ ID NO: 11]
5′-CATCTCCAGAGTCCAGCACA-3′; (Rev) [SEQ ID NO: 12]
mouse CTGF:
5′-AAAGTGCATCCGGACACCTAA-3′; (For) [SEQ ID NO: 13]
5′-TGCAGCCAGAAAGCTCAAACT-3′. (Rev) [SEQ ID NO: 14]
Human ets-2 protein-Accession number: AAP36808 [SEQ ID NO: 15]:
MNDFGIKNMDQVAPVANSYRGTLKRQPAFDTFDGSLFAVFPSLNEEQTL
QEVPTGLDSISHDSANCELPLLTPCSKAVMSQALKATFSGFKKEQRRLG
IPKNPWLWSEQQVCQWLLWATNEFSLVNVNLQRFGMNGQMLCNLGKERF
LELAPDFVGDILWEHLEQMIKENQEKTEDQYEENSHLTSVPHWINSNTL
GFGTEQAPYGMQTQNYPKGGLLDSMCPASTPSVLSSEQEFQMFPKSRLS
SVSVTYCSVSQDFPGSNLNLLTNNSGTPKDHDSPENGADSFESSDSLLQ
SWNSQSSLLDVQRVPSFESFEDDCSQSLCLNKPTMSFKDYIQERSDPVE
QGKPVIPAAVLAGFTGSGPIQLWQFLLELLSDKSCQSFISWTGDGWEFK
LADPDEVARRWGKRKNKPKMNYEKLSRGLRYYYDKNIIHKTSGKRYVYR
FVCDLQNLLGFTPEELHAILGVQPDTED
Mouse ets-2 protein Accession number: NP_035939 [SEQ ID NO: 16]
NDFGIKNMDQVAPVANSFRGTLKRQPAFDTFDGSLFAVLPSLSEDQTLQ
EVPTGLDSVSHDSASCELPLLTPCSKAVMSQALKATFSGFQKEQRRLGI
PKNPWLWSEQQVCQWLLWATNEFSLVNVNLHQFGMNGQMLCNLGKERFL
ELAPDFVGDILWEHLEQMIKENQEKTEDQYEENSHLNAVPHWINSNTLG
FSMEQAPYGMQAPNYPKDNLLDSMCPPSATPAALGSELQMLPKSRLNTV
NVNYCSISQDFPSSNVNLLNNNSGKPKDHDSPENGGDSFESSDSLLRSW
NSQSSLLDVQRVPSFESFEEDCSQSLCLSKLTMSFKDYIQERSDPVEQG
KPVIPAAVLAGFTGSGPIQLWQFLLELLSDKSCQSFISWTGDGWEFKLA
DPDEVARRWGKRKNKPKMNYEKLSRGLRYYYDKNIIHKTSGKRYVYRFV
CDLQNLLGFTPEELHAILGVQPDTED.

1. A marker for a fibrotic-related disorder, comprising an ets transcription factor, wherein the transcription factor comprises an ets-2 transcription factor or a phosphorylated ets-2 transcription factor.

2. The marker of claim 1, comprising a single amino acid mutation in ets-2 (threonine-72 to alanine).

3. An IFP-associated marker for a fibrotic-related disorder, comprising:
i) ets-2 transcription factor or a phosphorylated ets-2 transcription factor having a single amino acid mutation in ets-2 (threonine-72 to alanine); or,
ii) a linkage disequilibrium therewith wherein the allelic status in the subject is predictive of the subject's risk for having or developing the IPF-related disorder.

4. (canceled)

5. (canceled)

6. (canceled)

7. (canceled)

8. (canceled)

9. (canceled)

10. A kit for determining the levels of at least the marker of claim 1, characterized in that it comprises a specific probe for the marker.

11. A kit according to claim 10, characterized in that at least one of the probes is a specific antibody.

12. A kit according to claim 11, characterized in that it is a kit for one or more of: an immunoassay, an immunochromatography, and ELISA.

13. A kit according to claim 12, characterized in that it further comprises other components selected from secondary antibodies, tracers, buffers, diluents, standards, calibration controls, test cartridges, vials, chromatographic strips, microplates and instructions for use.

14. A kit according to claim 10, characterized in that it comprises packaging with a label with the indication for the evaluation of the presence and severity of fibrosis or an equivalent indication.

15. A kit according to claim 14, with the indication for the evaluation of the presence and severity of fibrosis.

16. A method for determining the severity of an inflammatory disorder comprising:
i) determining levels of transcription ets-2 in a subject, and
ii) comparing the levels of ets-2 to reference ets-2 concentrations that correlate to specific stages of an inflammatory disorder.

17. The method of claim 16, wherein the ets-2 levels are determined from a tissue sample obtained from the subject.

18. A method for assisting in the diagnosis of an inflammatory disorder or propensity of developing an inflammatory disorder in a subject comprising:
i) obtaining a biological sample from the subject; and,
ii) determining levels of ets-2 in the biological sample,
wherein altered levels of ets-2 in the biological sample relative to a control is indicative of an inflammatory disorder or an increased propensity for developing an inflammatory disorder.

19. A method of treatment of a chronic inflammatory disease in a subject, the method comprising administering to the subject an effective amount of an agent which beneficially alters the activity or expression of ets-2.

20. The method of claim 19, wherein the agent is selected from the group consisting of anti-ets-2 antibodies and fragments thereof and other agents which inhibit or block ets-2 activity, or fragments thereof.

21. The method of claim 19, wherein the agent is selected from the group consisting of antisense RNA targeted to the ets-2 gene, DNA constructs for expression of antisense RNA targeted to the ets-2 gene, ribozymes targeted to the ets-2 gene, DNA constructs for expression of ribozymes targeted to the ets-2 gene and, DNAzymes targeted to the ets-2 gene.

22. The method of claim 19, wherein the agent is selected from ets-2, functional fragments thereof, and ets-2 mimetic compounds.

23. The method of claim 18, wherein altering the activity comprises decreasing the activity of the transcription factor.

24. The method of claim 23, wherein the step of decreasing the activity of the transcription factor further comprises decreasing the function of the transcription factor or blocking the expression of the transcription factor.

25. The method of claim 18, wherein the chronic inflammatory disease is pulmonary fibrosis.

26. A method for determining the efficacy of a treatment for an inflammatory disorder in a subject comprising:
i) obtaining one or more biological samples from the subject during the course of the treatment; and
ii) comparing the levels of ets-2 in the samples to the levels of ets-2 in a biological sample obtained from the subject prior to treatment, or
comparing the levels of ets-2 in the samples to levels of ets-2 indicative of different stages of an inflammatory disease or disorder or autoimmune disease.

27. The method of claim 26, wherein the method includes modulating the expression one or more of: CCL12, active TGFβ, connective tissue growth factor (CTGF), type-I collagen, type-III collagen and α-smooth muscle actin (αSMA).

28. (canceled)

29. (canceled)

30. (canceled)

31. (canceled)

32. A pharmaceutical composition for the treatment of fibrosis comprising a compound that reduces the expression of transcription factor ets-2 and a pharmaceutically acceptable carrier.

33. The pharmaceutical composition of claim 32, wherein the compound is a small molecule, peptide, or antisense RNA.

34. A method for determining the aggressiveness of a fibrosis-related disorder comprising:
i) obtaining a sample from a subject having a fibrosis-related disorder; and
ii) detecting the presence of one or more ets transcription factors in the sample, the decreased presence of the transcription factor indicating that the fibrosis-related disorder is aggressive.

35. A method for predicting a subject's risk factors for an IPF-related disorder, the method comprising detecting the allelic status of one or more polymorphisms in a nucleic acid sample of the subject, wherein the polymorphism is one or more of:
i) an IFP-associated marker for a fibrotic-related disorder, comprising ets-2 transcription factor or a phosphorylated ets-2 transcription factor having a single amino acid mutation in ets-2 (threonine-72 to alanine); or,
ii) a linkage disequilibrium therewith wherein the allelic status in the subject is predictive of the subject's risk for having or developing the IPF-related disorder.

36. A method of screening a subject for a prognostic biomarker of an IPF-related disorder, comprising detecting the allelic status of one or more polymorphisms in a nucleic acid sample of the subject, wherein the polymorphism is one or more of:
i) an IFP-associated marker for a fibrotic-related disorder, comprising ets-2 transcription factor or a phosphorylated ets-2 transcription factor having a single amino acid mutation in ets-2 (threonine-72 to alanine); or,
ii) a linkage disequilibrium therewith wherein the allelic status in the subject is predictive of the subject's risk for having or developing the IPF-related disorder.

37. The method of claim 34, further comprising the step of correlating the allelic status of the polymorphism in the subject with the allelic status of the polymorphism in a reference population to predict the subject's risk for having or developing the IFP-related disorder.

38. The method of claim 34, further comprising the step of correlating the allelic status of the polymorphism in the subject with the allelic status of the polymorphism in a reference population to predict the prognostic outcome of the disorder in the subject.

39. The method of claim 34, further comprising the step of correlating the allelic status of the polymorphism in the subject with the allelic status of the polymorphism in a reference population to predict the subject's response to treatment.

40. A microarray comprising oligonucleotide probes capable of hybridizing under stringent conditions to one or more nucleic acid molecules having a polymorphic variant sequence at the site encoding the marker of claim 1.

41. The method of claim 35, further comprising the step of correlating the allelic status of the polymorphism in the subject with the allelic status of the polymorphism in a reference population to predict the subject's risk for having or developing the IFP-related disorder.

42. The method of claim 35, further comprising the step of correlating the allelic status of the polymorphism in the subject with the allelic status of the polymorphism in a reference population to predict the prognostic outcome of the disorder in the subject.

43. The method of claim 35, further comprising the step of correlating the allelic status of the polymorphism in the subject with the allelic status of the polymorphism in a reference population to predict the subject's response to treatment.

44. The method of claim 36, further comprising the step of correlating the allelic status of the polymorphism in the subject with the allelic status of the polymorphism in a reference population to predict the subject's risk for having or developing the IFP-related disorder.

45. The method of claim 36, further comprising the step of correlating the allelic status of the polymorphism in the subject with the allelic status of the polymorphism in a reference population to predict the prognostic outcome of the disorder in the subject.

46. The method of claim 36, further comprising the step of correlating the allelic status of the polymorphism in the subject with the allelic status of the polymorphism in a reference population to predict the subject's response to treatment.
